(19) United States
(12) Patent Application Publication
(10) Pub. No.: US 2015/0011481 A1
(43) Pub. Date: Jan. 8, 2015

(54) Methods for Treating HCV

(71) Applicant: AbbVie Inc., North Chicago, IL (US)

(72) Inventors: Regis A. Vilchez, Lake Forest, IL (US); Lino X. Rodrigues, JR., Evanston, IL (US); Barry M. Bernstein, Mequon, WI (US); Thomas J. Podsadecki, Northbrook, IL (US); Scott C. Brun, Green Oaks, IL (US); Daniel E. Cohen, Wilmette, IL (US); Rajeev M. Menon, Buffalo Grove, IL (US); Amit Khatri, Waukegan, IL (US); Sven Mensing, Mannheim (DE); Sandeep Dutta, Lincolnshire, IL (US); Walid M. Awni, Green Oaks, IL (US); Emily O. Dumas, Libertyville, IL (US); Cheri E. Klein, Northbrook, IL (US); Tolga Baykal, Libertyville, IL (US)

(21) Appl. No.: 14/320,843

(22) Filed: Jul. 1, 2014

Related U.S. Application Data
(60) Provisional application No. 61/842,256, filed on Jul. 2, 2013.
(60) Provisional application No. 61/862,229, filed on Aug. 5, 2013.
(60) Provisional application No. 61/862,824, filed on Aug. 6, 2013.
(60) Provisional application No. 61/895,700, filed on Oct. 25, 2013.
(60) Provisional application No. 61/918,245, filed on Dec. 19, 2013.
(60) Provisional application No. 61/932,520, filed on Jan. 28, 2014.
(60) Provisional application No. 61/952,453, filed on Mar. 13, 2014.
(60) Provisional application No. 61/972,855, filed on Mar. 31, 2014.
(60) Provisional application No. 62/008,778, filed on Jun. 6, 2014.

(51) Int. Cl. A61K 38/07; A61K 31/7056; A61K 31/427; A61K 38/05

(52) U.S. Cl. CPC A61K 38/07; A61K 38/05; A61K 31/7056; A61K 31/427
USPC 514/21.1; 514/21.91

(57) ABSTRACT

The present invention features interferon-free therapies for treating HCV genotype 1b, 2, 3 or 4. In one aspect, the therapies comprise administering Compound 1, ritonavir, and Compound 2 to a subject infected with HCV genotype 1b or 4, wherein the therapies do not include administration of any interferon, and the therapies last for 12 weeks. Preferably, the therapies do not include administration of any ribavirin.

### FIELD OF THE INVENTION

The present invention relates to interferon-free treatment for HCV.

### BACKGROUND OF THE INVENTION

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often incomplete. Therefore, there is a need for new therapies to treat HCV infection.

### BRIEF DESCRIPTION OF THE DRAWINGS

FIG. 1 shows the predicted median and 90% confidence interval of sustained virological response (SVR) percentage for different treatment durations of a 2-DAA regimen without ribavirin; wherein the 2 DAAs include (i) Compound 1 with ritonavir (Compound 1/r) and (ii) Compound 2.

### DESCRIPTION OF THE INVENTION

The present invention feature methods of treatment for HCV genotype (GT) 1b, 2, 3 or 4. The treatment comprises administering Compound 1 or a pharmaceutically acceptable salt thereof, and Compound 2 or a pharmaceutically acceptable salt thereof, to a patient infected with HCV genotype 1b, 2, 3, or 4. The treatment does not include administration of any interferon. To improve pharmacokinetics, Compound 1 or the salt thereof preferably is co-administered with ritonavir or another CYP3A4 inhibitor (e.g., cobicistat).

A treatment regimen of the invention generally constitutes a complete treatment, and no subsequent interferon-containing regimen is intended. Therefore, a treatment or use described herein generally does not include any subsequent interferon-containing treatment.

A treatment regimen of the invention preferably lasts no more than 12 weeks. More preferably, a treatment regimen of the invention lasts from 8 to 12 weeks, such as 8, 9, 10, 11, or 12 weeks. Highly preferably, a treatment regimen of the invention lasts for 12 weeks.

Compound 1

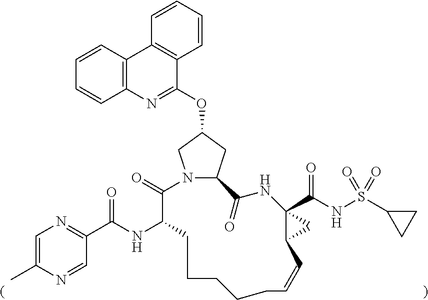 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

is also known as (2R,6S,13aS,14aR,16aS,Z)—N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. Compound 1 is a potent HCV protease inhibitor. The synthesis and formulation of Compound 1 are described in U.S. Patent Application Publication Nos. 2010/0144608 and 2011/0312973, both of which incorporated herein by reference in their entireties.

Compound 2

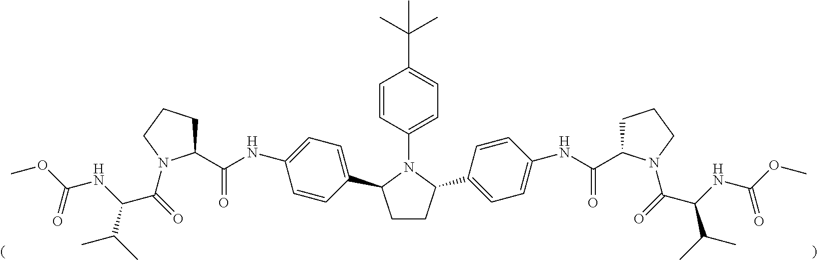

is also known as dimethyl (2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate. The preparation and formulation of Compound 2 are described in U.S. Patent Application Publication Nos. 2010/0317568 and 2012/0258909, both of which are incorporated herein by reference in their entireties.

In any method or treatment regimen of the invention, or any aspect, embodiment or example described herein, Compound 1 can be administered, for example, 100 mg once daily (QD), Compound 2 25 mg QD, and ritonavir 100 mg QD.

In any method or treatment regimen of the invention, or any aspect, embodiment or example described herein, Compound 1, ritonavir and Compound 2 can be, for example, co-formulated in a single dosage form. Preferably, Compound 1, ritonavir and Compound 2 are co-formulated in a single solid dosage form. More preferably, Compound 1, ritonavir and Compound 2 are each formulated in an amorphous solid dispersion comprising a hydrophilic polymer and a pharmaceutically acceptable surfactant. Compound 1, ritonavir and Compound 2 can be formulated in the same solid dispersion; Compound 1, ritonavir and Compound 2 can also be formulated in separate solid dispersions and then mixed together to provide a single solid dosage form.

In any method or treatment regimen of the invention, or any aspect, embodiment or example described herein, Compound 1, ritonavir and Compound 2 can be, for example, co-formulated in a single dosage form which comprises 75 mg Compound 1, 50 mg ritonavir, and 12.5 mg Compound 2.

In any method or treatment regimen of the invention, or any aspect, embodiment or example described herein, a treatment regimen of the invention can, for example, further comprise administering ribavirin to the patient. Preferably, in any method or treatment regimen of the invention, or any aspect, embodiment or example described herein, a treatment regimen of the invention does not include administration of any ribavirin.

In any method or treatment regimen of the invention, or any aspect, embodiment or example described herein, the patient can be a treatment-naïve patient, an interferon null responder, or an interferon non-responder.

In any method or treatment regimen of the invention, or any aspect, embodiment or example described herein, the patient can be a treatment-experienced patient (e.g., an interferon null responder or an interferon non-responder).

In any method or treatment regimen of the invention, or any aspect, embodiment or example described herein, the patient can be a non-cirrhotic, treatment-naïve patient.

In any method or treatment regimen of the invention, or any aspect, embodiment or example described herein, the patient can be a non-cirrhotic, treatment-experienced patient (e.g., an interferon null responder or an interferon non-responder).

In any method or treatment regimen of the invention, or any aspect, embodiment or example described herein, the patient can be a treatment-naïve patient with compensated cirrhosis.

In any method or treatment regimen of the invention, or any aspect, embodiment or example described herein, the patient can be a treatment-experienced patient (e.g., an interferon null responder or an interferon non-responder) with compensated cirrhosis.

In any method or treatment regimen of the invention, or any aspect, embodiment or example described herein, the patient can be an interferon null responder with compensated cirrhosis.

In any method or treatment regimen of the invention, or any aspect, embodiment or example described herein, the patient can be an interferon non-responder with compensated cirrhosis.

In any method or treatment regimen of the invention, or any aspect, embodiment or example described herein, the patient can be a patient without cirrhosis.

In any method or treatment regimen of the invention, or any aspect, embodiment or example described herein, the patient can be a cirrhotic patient.

In any method or treatment regimen of the invention, or any aspect, embodiment or example described herein, the patient can be a patient with compensated cirrhosis.

In any method or treatment regimen of the invention, or any aspect, embodiment or example described herein, Compound 1/r and Compound 2 can also be used in combination with Compound 3 (N-(6-(3-tert-butyl-5-(2,4-dioxo-3,4-dihydropyrimidin-1(2H)-yl)-2-methoxyphenyl)naphthalen-2-yl)methanesulfonamide) as described below.

In any method or treatment regimen of the invention, or any aspect, embodiment or example described herein, Compound 1/r and Compound 2 can be administered QD.

In any method or treatment regimen of the invention, or any aspect, embodiment or example described herein, Compound 1/r and Compound 2 can be administered QD; and if Compound 3 is also administered, Compound 3 can be administered BID.

In any method or treatment regimen of the invention, or any aspect, embodiment or example described herein, Compound 1/r and Compound 2 can be administered QD; and if Compound 3 is also administered, Compound 3 can be administered QD.

In any method or treatment regimen of the invention, or any aspect, embodiment or example described herein, the patient can be a patient infected with HCV GT 1.

In any method or treatment regimen of the invention, or any aspect, embodiment or example described herein, the patient can be a patient infected with HCV GT 1a.

In any method or treatment regimen of the invention, or any aspect, embodiment or example described herein, the patient can be a patient infected with HCV GT 1b.

In any method or treatment regimen of the invention, or any aspect, embodiment or example described herein, the patient can be a patient infected with HCV GT 4.

In any method or treatment regimen of the invention, or any aspect, embodiment or example described herein, the patient can be a patient infected with HCV GT 1 and without cirrhosis.

In any method or treatment regimen of the invention, or any aspect, embodiment or example described herein, the patient can be a patient infected with HCV GT 1a and without cirrhosis.

In any method or treatment regimen of the invention, or any aspect, embodiment or example described herein, the patient can be a patient infected with HCV GT 1b and without cirrhosis.

In any method or treatment regimen of the invention, or any aspect, embodiment or example described herein, the patient can be a patient infected with HCV GT 4 and without cirrhosis.

In any method or treatment regimen of the invention, or any aspect, embodiment or example described herein, the patient can be a patient infected with HCV GT 1 and with compensated cirrhosis.

In any method or treatment regimen of the invention, or any aspect, embodiment or example described herein, the patient can be a patient infected with HCV GT 1a and with compensated cirrhosis.

In any method or treatment regimen of the invention, or any aspect, embodiment or example described herein, the patient can be a patient infected with HCV GT 1b and with compensated cirrhosis.

In any method or treatment regimen of the invention, or any aspect, embodiment or example described herein, the patient can be a patient infected with HCV GT 4 and with compensated cirrhosis.

In one aspect, the present invention features methods of treatment for HCV genotype 1b. The treatment comprises administering Compound 1 or a pharmaceutically acceptable salt thereof, and Compound 2 or a pharmaceutically acceptable salt thereof, to a patient infected with HCV genotype 1b, wherein the treatment does not include administration of interferon to the patient. The treatment can last from 8 to 12 weeks. For example, the treatment can last for 8, 9, 10, 11 or 12 weeks. Preferably, the treatment lasts for 12 weeks.

Compound 1 preferably is co-administered with ritonavir. Another CYP3A4 inhibitor, such as cobicistat, can also be used in lieu of ritonavir.

In any method or treatment regimen of this aspect of the invention, the patient being treated can be a treatment-naïve patient.

In any method or treatment regimen of this aspect of the invention, the patient being treated can be a treatment-experienced patient

In any method or treatment regimen of this aspect of the invention, the patient being treated can be an interferon null responder.

In any method or treatment regimen of this aspect of the invention, the patient being treated can be an interferon non-responder.

In any method or treatment regimen of this aspect of the invention, the patient being treated can be a non-cirrhotic, treatment-naïve patient.

In any method or treatment regimen of this aspect of the invention, the patient being treated can be a non-cirrhotic, treatment-experienced patient

In any method or treatment regimen of this aspect of the invention, the patient being treated can be a non-cirrhotic, interferon null responder.

In any method or treatment regimen of this aspect of the invention, the patient being treated can be a non-cirrhotic, interferon non-responder.

In any method or treatment regimen of this aspect of the invention, the patient being treated can be a treatment-naïve patient with compensated cirrhosis.

In any method or treatment regimen of this aspect of the invention, the patient being treated can be a treatment-experienced patient with compensated cirrhosis.

In any method or treatment regimen of this aspect of the invention, the patient being treated can be an interferon null responder with compensated cirrhosis.

In any method or treatment regimen of this aspect of the invention, the patient being treated can be an interferon non-responder with compensated cirrhosis.

In any method or treatment regimen of this aspect of the invention, the patient can be a patient without cirrhosis.

In any method or treatment regimen of this aspect of the invention, the patient can be a cirrhotic patient.

In any method or treatment regimen of this aspect of the invention, the patient can be a patient with compensated cirrhosis

In this aspect of invention or any embodiment or example thereof, a treatment regimen can further comprise administering ribavirin to said patient. Preferably, in this aspect of invention or any embodiment or example thereof, a treatment regimen does not comprise administration of any ribavirin to said patient.

In another aspect, the present invention features methods of treatment for HCV genotype 4. The treatment comprises administering Compound 1 or a pharmaceutically acceptable salt thereof, and Compound 2 or a pharmaceutically acceptable salt thereof, to a patient infected with HCV genotype 4, wherein the treatment does not include administration of any interferon to the patient. The treatment can last from 8 to 12 weeks. For example, the treatment can last for 8, 9, 10, 11 or 12 weeks. Preferably, the treatment lasts for 12 weeks.

Compound 1 preferably is co-administered with ritonavir. Another CYP3A4 inhibitor, such as cobicistat, can also be used in lieu of ritonavir.

In any method or treatment regimen of this aspect of the invention, the patient being treated can be a treatment-naïve patient.

In any method or treatment regimen of this aspect of the invention, the patient being treated can be a treatment-experienced patient

In any method or treatment regimen of this aspect of the invention, the patient being treated can be an interferon null responder.

In any method or treatment regimen of this aspect of the invention, the patient being treated can be an interferon non-responder.

In any method or treatment regimen of this aspect of the invention, the patient being treated can be a non-cirrhotic, treatment-naïve patient.

In any method or treatment regimen of this aspect of the invention, the patient being treated can be a non-cirrhotic, treatment-experienced patient

In any method or treatment regimen of this aspect of the invention, the patient being treated can be a non-cirrhotic, interferon null responder.

In any method or treatment regimen of this aspect of the invention, the patient being treated can be a non-cirrhotic, interferon non-responder.

In any method or treatment regimen of this aspect of the invention, the patient being treated can be a treatment-naïve patient with compensated cirrhosis.

In any method or treatment regimen of this aspect of the invention, the patient being treated can be a treatment-experienced patient with compensated cirrhosis.

In any method or treatment regimen of this aspect of the invention, the patient being treated can be an interferon null responder with compensated cirrhosis.

In any method or treatment regimen of this aspect of the invention, the patient being treated can be an interferon non-responder with compensated cirrhosis.

In any method or treatment regimen of this aspect of the invention, the patient can be a patient without cirrhosis.

In any method or treatment regimen of this aspect of the invention, the patient can be a cirrhotic patient.

In any method or treatment regimen of this aspect of the invention, the patient can be a patient with compensated cirrhosis

Preferably, in this aspect of invention or any embodiment or example thereof, a treatment regimen comprises administering ribavirin to said patient. Alternatively, in this aspect of invention or any embodiment or example thereof, a treatment regimen does not include administration of any ribavirin to said patient.

As used herein, non-limiting examples of interferon include pegylated interferon (pegIFN), such as pegylated interferon-alpha-2a or pegylated interferon-alpha-2b. Specific examples of interferon include, but are not limited to, Pegasys, PegIntron, Roferon A, or Intron A. Specific examples of ribavirin (RBV) include, but are not limited to, Copegus, Rebetol, or Ribasphere.

GUIDANCE FOR INDUSTRY—CHRONIC HEPATITIS C VIRUS INFECTION: DEVELOPING DIRECT-ACTING ANTIVIRAL AGENTS FOR TREATMENT (FDA, September 2010, draft guidance) define treatment-naïve, partial responder, responder relapser (i.e., rebound), and null responder patients. The interferon non-responder patients include null responder, partial responder as well as rebound patients.

Various measures can be used to evaluate the responsiveness or effectiveness of an HCV treatment. One such measure is rapid virologic response (RVR), meaning that HCV is undetectable in the subject after 4 weeks of treatment. Another measure is early virologic response (EVR), meaning that the subject has >2 log₁₀ reduction in viral load after 12 weeks of treatment. Another measure is complete EVR (cEVR), meaning the HCV is undetectable in the serum of the subject after 12 weeks of treatment. Another measure is extended RVR (eRVR), meaning achievement of both RVR and cEVR, that is, HCV is undetectable at week 4 and 12. Another measure is the presence or absence of detectable virus at the end of therapy (EOTR). Another measure is SVR, which, as used herein, means that the virus is undetectable at the end of therapy and for at least 8 weeks after the end of therapy (SVR8); preferably, the virus is undetectable at the end of therapy and for at least 12 weeks after the end of therapy (SVR12); more preferably, the virus is undetectable at the end of therapy and for at least 16 weeks after the end of therapy (SVR16); and highly preferably, the virus is undetectable at the end of therapy and for at least 24 weeks after the end of therapy (SVR24). A desired treatment should achieve significantly high SVR rates.

Preferably, a treatment regimen of the invention achieves at least 80% SVR12 rate. More preferably, a treatment regimen of the invention achieves at least 90% SVR12 rate. Highly preferably, a treatment regimen of the invention achieves at least 95% SVR12 rate.

A treatment regimen of the invention may also comprise administering to the patient one or more other HCV direct acting agents (DAAs), such as other HCV protease inhibitors, HCV polymerase inhibitors, other HCV NS5A inhibitors, cyclophilin inhibitors, or combinations thereof.

Non-limiting examples of HCV protease inhibitors include telaprevir (Vertex), boceprevir (Merck), BI-201335 (Boehringer Ingelheim), GS-9451 (Gilead), and BMS-650032 (BMS). Other suitable protease inhibitors include, but are not limited to, ACH-1095 (Achillion), ACH-1625 (Achillion), ACH-2684 (Achillion), AVL-181 (Avila), AVL-192 (Avila), BMS-650032 (BMS), danoprevir (RG7227/ITMN-191, Roche), GS-9132 (Gilead), GS-9256 (Gilead), IDX-136 (Idenix), IDX-316 (Idenix), IDX-320 (Idenix), MK-5172 (Merck), narlaprevir (Schering-Plough Corp), PHX-1766 (Phenomix), TMC-435 (Tibotec), vaniprevir (MK-7009, Merck), VBY708 (Virobay), VX-500 (Vertex), VX-813 (Vertex), and VX-985 (Vertex).

Non-limiting examples of non-nucleoside HCV polymerase inhibitors include GS-9190 (Gilead), BI-207127 (Boehringer Ingelheim), and VX-222 (VCH-222) (Vertex & ViraChem). Non-limiting examples of nucleotide HCV polymerase inhibitors include GS-7977 (Gilead). Other suitable, non-limiting examples of HCV polymerase inhibitors include ANA-598 (Anadys), BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-759 (Vertex), GS-6620 (Gilead), IDX-102 (Idenix), IDX-184 (Idenix), INX-189 (Inhibitex), MK-0608 (Merck), RG7128 (Roche), TMC64912 (Medivir), GSK625433 (GlaxoSmithKline), BCX-4678 (BioCryst), ALS-2200 (Alios BioPharma/Vertex), and ALS-2158 (Alios BioPharma/Vertex).

Non-limiting examples of NS5A inhibitors include BMS-790052 (BMS) and GS-5885 (Gilead). Other non-limiting examples of suitable NS5A inhibitors include GSK62336805 (GlaxoSmithKline), ACH-2928 (Achillion), AZD2836 (Astra-Zeneca), AZD7295 (Astra-Zeneca), BMS-790052 (BMS), BMS-824393 (BMS), GS-5885 (Gilead), PPI-1301 (Presidio), PPI-461 (Presidio) A-831 (Arrow Therapeutics), and A-689 (Arrow Therapeutics).

Non-limiting examples of cyclophilin inhibitors include alisporovir (Novartis & Debiopharm), NM-811 (Novartis), and SCY-635 (Scynexis).

Compound 1 (or a pharmaceutically acceptable salt thereof) and Compound 2 (or a pharmaceutically acceptable salt thereof) can be used to treat HCV patients with cirrhosis. The patients can infected with HCV genotypes 1, 2, 3, 4, 5 or 6, such as genotype 1a or 1b, and the cirrhosis can be either compensated or decompensated. The methods comprise administering Compound 1 or a pharmaceutically acceptable salt thereof, and Compound 2 or a pharmaceutically acceptable salt thereof, to such a patient, wherein the treatment does not include administration of interferon to the patient. The treatment can last from 8 to 12 weeks; for example, the treatment can last for 8, 9, 10, 11 or 12 weeks. Preferably, the treatment lasts for 12 weeks. Longer treatment durations can also be used, such as 24 weeks or a less duration. Ribavirin can be administered; or alternatively, the treatment does not include administering ribavirin. Preferably, the treatment further comprises administering ribavirin and N-(6-(3-tert-butyl-5-(2,4-dioxo-3,4-dihydropyrimidin-1(2H)-yl)-2-methoxyphenyl)naphthalen-2-yl)methanesulfonamide (or a pharmaceutically acceptable salt thereof). See U.S. Patent Application Publication No. 2013/0102525. To improve pharmacokinetics, Compound 1 or the salt thereof preferably is co-administered with ritonavir or another CYP3A4 inhibitor (e.g., cobicistat). Other known DAA combinations that are currently being tested in clinical trials can also be used to treat cirrhotic patients in similar regimens.

It should be understood that the above-described embodiments and the following examples are given by way of illustration, not limitation. Various changes and modifications within the scope of the present invention will become apparent to those skilled in the art from the present description.

### Example 1

### Interferon- and Ribavirin-Free Treatment of HCV Genotype 1b

Treatment-naïve patients and prior pegIFN/RBV null responders received Compound 1 (150 mg QD), ritonavir (100 mg QD) and Compound 2 (25 mg QD) for 12 weeks. 42 treatment-naïve patients and 40 prior pegIFN/RBV null responders with chronic HCV genotype 1b infection were enrolled. All patients are non-cirrhotic. Baseline characteristics are shown in Table 1. Observed rates of HCV RNA <25 IU/mL (detection limit) at treatment weeks 4 and 12 of the treatment, as well as observed SVR₄ rates (percent of patients with HCV RNA <25 IU/mL at post-treatment week 4) are summarized in Table 1. SVR₄ rate was 100% among treatment-naïve patients and 87.9% among prior null responders.

Further follow-up showed that among the 39 treatment-naïve patients that were actually tested at post-treatment week 8, 100% of the patients did not have detectable HCV RNA; and among the 30 treatment-naïve patients that were actually tested at post-treatment week 12, 97% of the patients (29/30) did not have detectable HCV RNA. Follow-up testing showed that among the 42 treatment-naïve patients, 40 patients achieved SVR₁₂, and the two remaining patients did not achieve SVR₁₂ due to loss to follow-up.

Testing also showed that among the 39 null responders that were actually tested at post-treatment week 4, 90% of the patients (35/39) did not have detectable HCV RNA. Further testing at post-treatment week 8 showed that 87% of the null responders that were actually tested (26/30) did not have detectable HCV RNA. Follow-up testing showed that among the 40 prior pegIFN/RBV null responders, 36 patients achieved SVR₁₂.

Among the 82 patients, there were no discontinuations due to adverse events (AE) or laboratory abnormalities. There were 2 serious AEs (both not related to study drug). Two subjects interrupted study drug due to AEs. One interruption was probably related to study drug (increased ALT, AST, and bilirubin); these values improved during resumed treatment or after completion.

TABLE 1

| | |
|---|---|
| | Prior Null |
| Treatment-naïve Patients | Responders |
| (N = 42) | (N = 40) |

Baseline characteristics

| | | | | |
|---|---|---|---|---|
| Male, n (%) | 25 | (59.5) | 15 | (37.5) |
| White race, n (%) | 27 | (65.9) | 39 | (97.5) |
| Age <50 yr, n (%) | 7 | (16.7) | 13 | (32.5) |
| Weight <85 kg, n (%) | 27 | (64.3) | 28 | (70.0) |
| IL28B CC, n (%) | 13 | (31.7) | 2 | (5.0) |

Efficacy

| | | | | |
|---|---|---|---|---|
| HCV RNA <25 IU/mL at | 42/42 | (100) | 39/40 | (97.5) |
| treatment week 4, n/N (%)* | | | | |
| HCV RNA <25 IU/mL at | 40/40 | (100) | 39/40 | (97.5) |
| treatment week 12, n/N (%)* | | | | |
| SVR₄, n/N (%)* | 39/39 | (100) | 29/33 | (87.9) |

| | | |
|---|---|---|
| On-treatment failure, n | 0 | 1 |
| Relapse, n | 0 | 3 |

*Observed data. Excludes patients with data missing for reasons besides virologic failure

### Example 2

### Clinical Modeling for Interferon-free Treatment of HCV Genotype 4

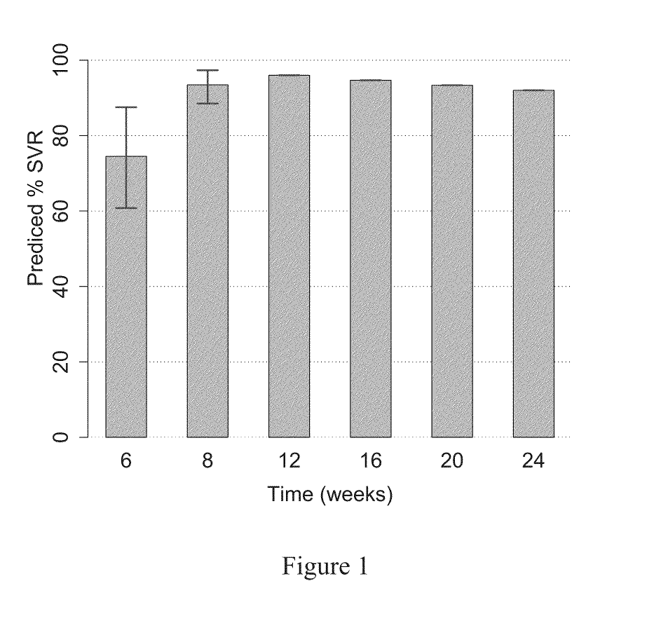

A novel clinical model for evaluating appropriate doses and durations of interferon-free HCV therapies using combinations of DAAs has been described in Example 6 of U.S. Patent Application Publication No. 2013/0102525, which example is incorporated herein by reference. Data from clinical studies, as well as in vitro replicon experiments, of Compound 1 and Compound 2 were used for estimating the pharmacokinetic and viral dynamic model parameters. In vivo parameters for genotype 4 were approximated using in vitro data, based on the relationship between the in vivo and in vitro data for genotype 1. The model predicts that following 8 or 12 weeks of dosing with the combination of Compound 1 (150 mg QD), ritonavir (100 mg QD) and Compound 2 (25 mg QD), over 90% of genotype 4 treatment-naïve patients can achieve SVR. See FIG. 1. FIG. 1 shows the predicted median SVR percentage (“% SVR”) and 90% confidence interval (the vertical bar at the top of each SVR percentage column) for different treatment durations using a combination of Compound 1, ritonavir and Compound 2, without interferon. Similar or better SVR rates are expected when ribavirin is included in the regimen.

### Example 3

### Clinical Study of Interferon-Free Treatment of HCV Genotype 4

A clinical study of interferon-free treatment of HCV genotype 4 was conducted. Two groups of treatment naïve patients with HCV GT 4 infection were enrolled in the study, each group including about 40 patients. Compound 1 (150 mg QD), ritonavir (100 mg QD), and Compound 2 (25 mg QD) were administered to each patient in both groups. Weight-based Ribavirin was also administered to the patients in the first group, but not to the second group. The baseline characteristics of these patients are summarized in Table 2.

After 12-week treatment, the first group of patients (with ribavirin) achieved about 100% SVR12 rate, and the second group (without ribavirin) achieved about 90% SVR12.

TABLE 2

| | | | Treatment-naive | |
|---|---|---|---|---|
| | Treatment-naïve Patients | | Patients (Compound 1/ | |
| | (Compound 1/ritonavir + | | ritonavir + | |
| | Compound 2) | | Compound 2 + | |
| | (N = 44) | | Ribavirin)(N = 42) | |
| Male, n (%) | 24 | (54.5) | 27 | (64.3) |
| White race, n (%) | 37 | (84.1) | 38 | (90.5) |
| IL28B CC, n (%) | 12 | (27.3) | 11 | (26.2) |
| Fibrosis stage, | 5 | (11.6)* | 9 | (21.4) |
| ≧F2, n (%) | | | | |
| Baseline HCV | 6.07 | (0.62) | 6.12 | (0.58) |
| RNA level, log₁₀ | | | | |
| IU/mL, mean (SD) | | | | |
| RVR, n/N (%) | 43/43 | (100) | 41/42 | (97.6)** |
| EOTR, n/N (%) | 42/43 | (97.7) | 42/42 | (100) |

| | | |
|---|---|---|
| Breakthrough | 1 | 0 |

*Fibrosis score was missing for one patient in this group.
**One patient did not have HCV RNA suppressed below 25 IU/mL until Week 6. This patient did not achieve RVR, but achieved EOTR.

In another arm, 49 interferon partial/null responders or relapsers with HCV GT 4 infection were enrolled and treated with Compound 1 (150 mg QD), ritonavir (100 mg QD), Compound 2 (25 mg QD) and ribavirin for 12 weeks. The SVR4 for this group of patients was 100%. Seven (7) of the 49 patients were tested at post-treatment week 12, and the SVR12 was 100%.

Further analysis showed that Compound 1/ritonavir+ Compound 2, either with or without ribavirin, achieved high SVR rate among patients with different GT 4 subtypes. Accordingly, in any method or treatment regimen of the invention for treating GT 4, or any aspect, embodiment or example described herein for treating GT 4, identification of specific GT4 subtype prior to the initiation of therapy is optional. For example, in any method or treatment regimen of the invention for treating GT 4, or any aspect, embodiment or example described herein for treating GT 4, the method preferably does not comprise the identification of specific GT4 subtype prior to the initiation of therapy.

### Example 4

### Clinical Study of Interferon-Free Treatment of HCV Genotype 1b

This study was a double-blind controlled trial. Subjects were randomized (1:1) to 12 weeks of treatment with Compound 1 (150 mg QD), ritonavir (100 mg QD), Compound 2 (25 mg QD), and Compound 3 (250 mg BID), with weight-based ribavirin (1000 mg or 1200 mg daily divided BID, Arm A) or placebo for ribavirin (Arm B). Compound 3 is N-(6-(3-tert-butyl-5-(2,4-dioxo-3,4-dihydropyrimidin-1(2H)-yl)-2-methoxyphenyl)naphthalen-2-yl)methanesulfonamide

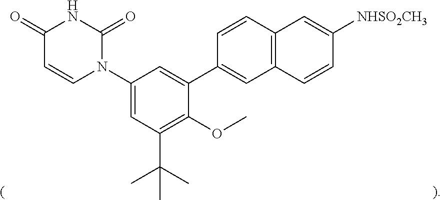

See International Application Publication No. WO2009/039127.

419 subjects received the above regimen, baseline characteristics as shown in Table 3. These subjects were infected with HCV GT 1b, and were treatment-naïve and non-cirrhotic. SVR12 rates (intent-to-treat) were 99.5% (Arm A) and 99.0% (Arm B) with no on-treatment virologic failure or post-treatment relapse among subjects receiving the above regimen without ribavirin. 19 subjects in Arm A and 0 in Arm B (P<0.001) had hemoglobin <10 g/dL. The most common adverse events in Arms A and B were headache (24.3% vs. 23.4%, P=NS) and fatigue (21.4% vs. 23.0%, P=NS.) No subjects discontinued due to adverse events.

TABLE 3

| | Arm A | Arm B |
|---|---|---|
| | (with RBV) | (without RBV) |
| | N = 210 | N = 209 |
| Male, n (%) | 106 (50.5) | 86 (41.1) |
| White race, n (%) | 198 (94.3) | 196 (94.2) |
| Age, mean (SD) | 48.4 (11.9) | 49.2 (12.0) |
| IL28B CC, n (%) | 44 (21.0) | 44 (21.1) |
| Baseline HCV RNA, log₁₀ IU/mL, | 6.29 (0.77) | 6.33 (0.67) |
| mean (SD) | | |
| SVR₁₂, n (%) | 209 (99.5) | 207 (99.0) |
| On-treatment virologic failure | 1 (0.5) | 0 |
| Relapse by post-treatment Week 12 | 0 | 0 |
| Missing SVR₁₂ data | 0 | 2 (1.0) |

This study shows that the combination of Compound 1/r, Compound 2 and Compound 3 is highly efficacious and safe with or without RBV for the treatment of HCV GT-1b infection. Both regimens were noninferior and superior compared to the historical rate for telaprevir+pegIFN/RBV. The addition of RBV appears not to provide additional clinical benefit for this GT-1b population when treated with Compound 1/r, Compound 2 and Compound 3

### Example 5

### Clinical Study of Interferon-free Treatment of HCV Genotype 1b

This example describes a phase 3 open-label study in HCV GT1b-infected patients who were randomized 1:1 to receive Compound 1 (150 mg QD) dosed with ritonavir (100 mg QD), Compound 2 (25 mg QD), and Compound 3 (250 mg BID) with RBV (Arm A) or without RBV (Arm B) for 12 weeks. 12-week post-treatment SVR rates (SVR12) for each treatment arm were compared to a historical telaprevir plus pegIFN/RBV threshold. Adverse events (AEs) were recorded for all patients receiving at least 1 dose of study drug. All patients were non-cirrhotic.

Of 187 treatment-experienced, randomized GT1b-infected patients, 186 were dosed with study drug and included in safety analyses; 179 patients received Compound 1/r and Compound 2 co-formulated drug and were included in intent-to-treat (ITT) efficacy analyses. In the ITT population, 35.2% were null-responders, 28.5% partial responders, and 36.3% relapsers to previous pegIFN/RBV treatment. Mean age (54.2 vs. 54.2 years), sex (49.5% vs. 60.0% male), and IL28B genotype CC (11.0% vs. 7.4%) were comparable between Arms A and B, respectively. After 12 weeks of treatment, intent-to-treat SVR₁₂ rates were 96.6% for Arm A and 100% for Arm B (Table 4). Similarly high SVR12 rates were observed in null-responders, partial responders, and relapsers. No patients experienced virologic failure; 2 patients in Arm A discontinued drug due to AEs. Adverse events were generally mild and the most frequent AEs were fatigue (31.9% vs. 15.8%, P=0.015), headache (24.2% vs. 23.2%, P>0.05), and nausea (20.9% vs 6.3%, P=0.005) in Arms A and B, respectively. The proportions of patients with hemoglobin below the lower limit of normal at the end of treatment and bilirubin >3× upper limit of normal were higher in patients receiving RBV; only 1.1% (2/186) of patients experienced hemoglobin <10 g/dL, both in Arm A.

TABLE 4
Efficacy and Safety of Compound 1/r/Compound 2/Compound 3 (3D) ±
RBV assessed on the ITT and safety population, respectively, n (%)

| | Arm A | Arm B |
|---|---|---|
| | 3D + RBV | 3D |
| Efficacy | (N = 88) | (N = 91) |
| SVR₁₂ | 85 (96.6) | 91 (100) |
| On-treatment virologic failure | 0 (0) | 0 (0) |
| Relapse by post-treatment Week 12 | 0 (0) | 0 (0) |
| Study drug discontinuation | 2 (2.3) | 0 (0) |
| Missing SVR₁₂ data | 1 (1.1) | 0 (0) |
| Safety | (N = 91) | (N = 95) |
| Treatment-emergent AEs | 72 (79.1) | 74 (77.9) |
| Serious AEs | 2 (2.2) | 2 (2.1) |
| AEs leading to drug discontinuation | 2 (2.2) | 0 (0) |
| Laboratory abnormalities of interest | | |
| Hemoglobin decrease to below LLN^{a} | 38 (42.0)*** | 5 (5.5) |
| Total bilirubin >3X ULN | 8 (8.8)** | 0 (0) |
| Alanine aminotransferase >5X ULN | 0 (0) | 0 (0) |

^{a}Secondary efficacy endpoint, thus using the ITT population, N's = 88 and 91 for Arm A and B, respectively. RBV, ribavirin; SVR₁₂, 12-week sustained virologic response; AEs, adverse events; LLN, lower limit of normal; ULN, upper limit of normal.
** and *** denote statistical significance at the .01 and .001 levels, respectively, using Fisher's exact test.

This study shows that a 12-week regimen of Compound 1/r, Compound 2 and Compound 3 with or without RBV achieved high rates of SVR12 (96.6% with RBV, and 100% with ribavirin) and was generally well tolerated, as evidenced by the low rate of treatment discontinuation and serious adverse events. The regimen without RBV was associated with lower rates of laboratory abnormalities including bilirubin elevation and hemoglobin decrease.

### Example 6

### Clinical Study of Interferon-Free Treatment of HCV Genotype 1a

HCV genotype 1a-infected, treatment-naïve patients in this study were randomized 1:2 to receive either blinded ribavirin twice daily at a dose of 1000 to 1200 mg per day according to body weight (1000 mg if body weight was <75 kg, 1200 mg if body weight was ≧75 kg) (Group A) or matching placebo (Group B) for 12 weeks. All patients received open-label Compound 1/r/Compound 2 (150 mg/100 mg/25 mg once daily) and Compound 3 (250 mg twice daily) for 12 weeks. Patients were followed for 48 weeks after the treatment period. A total of 305 patients were randomized and received at least one dose of study drug. Baseline demographics and characteristics were representative of typical North American or European GT 1a-infected HCV populations. All patients were non-cirrhotic.

After 12 weeks of treatment with Compound 1/r, Compound 2 and Compound 3, the sustained virologic response rate 12 weeks after treatment (SVR12) was 97.0% (97/100) in Group A, and 90.2% in Group B. SVR12 rates for Group A and Group B were both noninferior and superior to the historical rate for telaprevir plus peginterferon/ribavirin in treatment-naïve HCV genotype 1a-infected adults without cirrhosis.

The test for heterogeneity did not show a significant difference in SVR for sex, Hispanic or Latino ethnicity, age, fibrosis, viral load and IL28B genotype. SVR12 rates of at least 95% for both treatment arms were observed in certain subgroups, including patients with IL28B CC genotype (100% in Group A vs. 97% in Group B) and female patients (100% in Group A vs. 95% in Group B). Treatment differences between Group A and Group B did not vary significantly among the subgroups evaluated.

### Example 7

### Clinical Study of Interferon-Free Treatment of HCV Genotype 1

In this study, patients with Child-Pugh A cirrhosis were treated with Compound 1/r/Compound 2 (150 mg/100 mg/25 mg once daily), Compound 3 (250 mg twice daily), and weight-based ribavirin for 12 weeks. The primary efficacy analysis was the proportion of subjects achieving SVR12 compared to the historic telaprevir-based thresholds of 43% (non-inferiority) and 54% (superiority).

Eligible patients were adults 18 to 70 years old with chronic HCV genotype 1 infection and plasma HCV RNA level >10,000 IU/mL who were treatment-naïve or previously treated with peginterferon/ribavirin. All patients had cirrhosis, documented using liver biopsy or FibroScan, defined as compensated by a Child-Pugh class A score of <7 at screening, and no current or past clinical evidence of Child-Pugh B or C classification.

Patients were stratified as treatment-experienced or treatment-naïve according to previous treatment with peginterferon/ribavirin. Treatment-experienced patients were stratified by HCV subtype and by type of non-response to previous peginterferon/ribavirin treatment: null-responder, partial responder, or relapser. During the treatment period, patients received co-formulated Compound 1/r/Compound 2 (150 mg/100 mg/25 mg once daily), together with Compound 3 (250 mg twice daily) and ribavirin (1000 mg to 1200 mg divided twice daily, according to body weight), for 12 weeks.

After 12-week treatment according to the above-described regimen, the SVR12 rate was 91.8% (191 patients achieved SVR12 among a total of 208 patients studied). Table 5 summarizes the SVR12 rates among different patient populations. The SVR12 rate was noninferior and superior to the historic telaprevir plus peginterferon/ribavirin thresholds in HCV genotype 1 infected patients with cirrhosis.

At the end of the 12-week treatment, liver enzymes were normalized in most patients with baseline elevations. Activated partial thromboplastin time was normalized at the end of treatment in 47/67 (70.1%) patients with values >ULN at baseline. Mean total bilirubin values decreased to the end of treatment, and normalized post-treatment. In sum, the 12-week treatment resulted in high SVR rates and normalization of liver-related chemistry and coagulation profile abnormalities often present in patients with cirrhosis.

TABLE 5
SVR12 Rates after 12-Week Treatment

| | Patients Achieved SVR12/Total Patients |
|---|---|
| | (Percent) |
| GT1a by prior treatment | |
| response | |
| Naïve | 59/64 (92.2%) |
| Prior null responder | 40/50 (80.0%) |
| Prior partial responder | 11/11 (100%) |
| Prior relapser | 14/15 (93.3%) |
| GT1b by prior treatment | |
| response | |
| Naïve | 22/22 (100%) |
| Prior null responder | 25/25 (100%) |
| Prior partial responder | 6/7 (85.7%) |
| Prior relapser | 14/14 (100%) |

Naïve: Never received peginterferon/ribavirin for the treatment of HCV.
Prior null responder: Received at least 12 weeks of peginterferon/ribavirin for the treatment of HCV and failed to achieve a 2 log₁₀ IU/mL reduction in HCV RNA at week 12; or received at least 4 weeks of peginterferon/ribavirin for the treatment of HCV and achieved a <1 log₁₀ IU/mL reduction in HCV RNA at Week 4 (≧25 days).
Prior partial responder: Received at least 20 weeks of peginterferon/ribavirin for the treatment of HCV and achieved ≧2 log₁₀ reduction in HCV RNA at week 12, but failed to achieve HCV RNA undetectable at the end of treatment.
Prior relapser: Received at least 36 weeks of peginterferon/ribavirin for the treatment of HCV and was undetectable at or after the end of treatment, but HCV RNA was detectable within 52 weeks of treatment follow-up.

### Example 8

### Clinical Study of Interferon-Free Treatment of HCV Genotype 1

In this randomized, double-blind, placebo-controlled, multicenter trial, 631 treatment-naïve, non-cirrhotic HCV genotype 1-infected patients were assigned (3:1) to active regimen (Arm A; 473 patients) or matching placebos (Arm B; 158 patients). Arm A included administration of co-formulated Compound 1/r/Compound 2 (150 mg/100 mg/25 mg once daily), together with Compound 3 (250 mg twice daily) and weight-based ribavirin (1000 mg daily if body weight was <75 kg, 1200 mg daily if body weight was ≧75 kg), during a 12-week double-blind period. Arm B patients received matching placebos during this period. Ribavirin dose was modified due to adverse events in 5.5% of Arm A patients.

The primary endpoint was sustained virologic response 12 weeks post-treatment (SVR12). The primary analysis compared the response rate for Arm A with a historical control response rate for non-cirrhotic treatment-naïve patients who received telaprevir and peginterferon/ribavirin. Randomization was stratified by HCV subtype (1a, non-1a) and IL28B genotype (CC, non-CC).

The modified intention-to-treat SVR12 rate was 96.2% for Arm A (455 patients among the total of 473 Arm A patients achieved SVR12). This rate was noninferior and superior to the historical control SVR rate for telaprevir plus peginterferon/ribavirin. The SVR12 rate was 95.3% (307/322) in patients infected with HCV genotype 1a and 98.0% (148/151) in patients infected with HCV genotype 1b. These rates were superior to the historical control SVR rates for the respective subgroups. SVR12 rates were similarly high regardless of characteristics including IL28B genotype (CC: 96.5%, non-CC: 96.0%), race (Black: 96.4%, non-Black: 96.2%), baseline fibrosis score (F0-F1: 97.0%, F2: 94.3%, ≧F3: 92.5%), or baseline HCV RNA level (<800,000 IU/mL: 98.1%, ≧800,000 IU/mL: 95.7%). The SVR12 rate in patients with ribavirin dose modification was 93.5% (29/31) versus 96.4% (426/442) in those without modification. Even among patients with body-mass index ≧30 kg/m², the SVR12 rate was high (91.5%).

### Example 9

### Clinical Study of Interferon-Free Treatment of HCV Genotype 1

In this phase 3 clinical study, 394 patients were randomized (3:1) to active regimen or placebo during a 12-week double-blind period. The randomization schedule was stratified by type of response to previous peginterferon/ribavirin treatment (relapse, partial response, or null-response) and HCV subgenotype (1a, non-1a). During the double-blind period, patients randomized to active regimen received oral co-formulated Compound 1/r/Compound 2 (150 mg/100 mg/25 mg once daily), together with Compound 3 (250 mg twice daily) and weight-based ribavirin (1000 mg daily if body weight was <75 kg, 1200 mg daily if body weight was ≧75 kg; both divided twice daily), for 12 weeks. Patients randomized to placebo received matching placebo pills during this period. Treatment assignment was blinded to the investigator, patient, and sponsor during the double-blind period. All patients enrolled in the study were non-cirrhotic, peginterferon/ribavirin dual therapy-experienced, HCV genotype 1-infected patients with prior relapse (HCV RNA undetectable at end of treatment, but detectable thereafter), or partial (≧2 log₁₀IU/mL HCV RNA reduction at treatment week 12 but detectable at end of treatment) or null-response (<2 log₁₀IU/mL or <1 log₁₀IU/mL HCV RNA reduction at treatment week 12 or 4, respectively).

The primary endpoint was sustained virologic response 12 weeks post-treatment (SVR12). The primary efficacy analysis compared this rate in active regimen recipients to a historical response rate in HCV genotype 1-infected, non-cirrhotic, treatment-experienced patients who received telaprevir and peginterferon/ribavirin.

Among patients on active regimen, the SVR12 rate was 96.3% (286 of 297 patients on active regimen achieved SVR12). This was noninferior and superior to the historical control SVR rate for telaprevir and peginterferon/ribavirin. SVR12 rates among HCV-infected patients with HCV subtype 1a and 1b were 96.0% (166/173) and 96.7% (119/123), respectively. HCV subtype could not be determined for one patient, who achieved SVR12. The SVR12 rates were 95.3% (82/86) among prior relapsers, 100% (65/65) among partial responders, and 95.2% (139/146) among null-responders. SVR12 rates were also high across subgroups differing in characteristics including race, age, fibrosis score, and IL28B genotype.

Seven of the 293 patients (2.4%) experienced post-treatment viral relapse. At the time of relapse, 6 of the 7 patients had at least one variant known to confer resistance to one of the three direct-acting antivirals included in the regimen. The most frequently detected variants in the 5 genotype 1a-infected patients at the time of virologic failure were D168V (⅖) in NS3, M28V (⅗) and Q30R (⅖) in NS5A, and S556G (⅖) in NS5B. At the time of virologic failure, one of the genotype 1b-infected patients had no resistance-associated variants in NS3, NS5A or NS5B; the other genotype 1b-infected patient had Y56H and D168A in NS3, Y93H in NS5A and C316N+S556G in NS5B.

### Example 10

### Clinical Study of Interferon-Free Treatment of HCV Genotype 2

In this study, 37 non-cirrhotic, peginterferon/ribavirin (pegIFN/RBV) treatment-experienced Japanese adults with chronic HCV GT2 infection were treated with Compound 1/r (100 mg/100 mg or 150 mg/100 mg; QD) and Compound 2 (QD) for 12 weeks. These treatment-experienced patients included null responders, partial responders, and/or relapsers.

The SVR12 and SVR24 rates for the Compound 1/r (100 mg/100 mg) arm were 57.9% (N=19), and for the Compound 1/r (150 mg/100 mg) arm were 72.2% (N=18). Two of 8 GT2b-infected patients treated with Compound 1/r (100 mg/100 mg) plus Compound 2 achieved SVR24; three of 8 GT2b-infected patients treated with Compound 1/r (150 mg/100 mg) plus Compound 2 achieved SVR24; nine of 11 GT non-2b-infected patients treated with Compound 1/r (100 mg/100 mg) plus Compound 2 achieved SVR24; and all ten GT2b-infected patients treated with Compound 1/r (150 mg/100 mg) plus Compound 2 achieved SVR24.

### Example 11

### Clinical Study of HCV GT1 Infected Patients Receiving Chronic Opioid 1 Therapy

Non-cirrhotic patients with chronic HCV GT1 infection who were on stable methadone or buprenorphine+/− naloxone therapy were enrolled in this open-label study. Patients were treated for 12 weeks with co-formulated Compound 1/r/Compound 2 (2 tabs QD), Compound 3 (1 tab BID), and weight-based RBV (3D+RBV). The percentage of patients achieving SVR12 (HCV RNA <LLOQ 12 weeks post-treatment) was assessed in an intent-to-treat analysis.

38 patients were enrolled (19 on methadone, 19 on buprenorphine). Mean age was 48.2 years, 66% were male, 95% were treatment-naïve, 84% had GT1a infection, and 68% had IL28b non-CC genotype. One patient prematurely discontinued due to serious adverse events unrelated to study drug (cerebrovascular accident and sarcoma). The remaining 37 subjects (97.4%) all achieved SVR12. There were no virologic failures. The most frequent adverse events were nausea (50%), fatigue (47.4%), and headache (31.6%); 8 patients experienced hemoglobin <10 g/dL while on treatment, which was managed with RBV dose reduction. No dose adjustments of methadone or buprenorphine were reported. Among patients on stable methadone or buprenorphine therapy, the 3D+RBV regimen was well tolerated and achieved an SVR12 rate of 97.4%.

Another study also showed that the 3D regimen with or without RBV was well tolerated in patients on chronic opioid substitution treatment with methadone or buprenorphine, with a high SVR12 rate of over 95%.

### Example 12

### Clinical Study of Patients Co-Infected with Hepatitis C and HIV-1

This was a randomized, open-label study evaluating the 3D+RBV regimen for 12 weeks. Study eligibility included: HCV treatment-naïve or pegIFN/RBV-experienced, presence or absence of cirrhosis (Child-Pugh A), CD4+ count ≧200 cells/mm³ or CD4+%>14%, and plasma HIV-1 RNA suppressed on a stable atazanavir- or raltegravir-inclusive antiretroviral regimen. The primary endpoint is SVR 12 weeks post-treatment (SVR12). The baseline characteristics of the patients are summarized in Table 6.

Virologic response at end-of-treatment (EOTR) and 4 weeks post-treatment (SVR4) was achieved by 30/31 (96.8%) and 29/31 (93.5%) patients, respectively. One patient withdrew consent prior to finishing treatment but had an undetectable HCV RNA at last study visit (week 10), and another patient experienced virologic relapse at post-treatment week 2. No patient experienced a serious AE or discontinued study drugs due to an AE. Elevation in total bilirubin was the most common laboratory abnormality, predominantly in patients receiving atazanavir. HIV-1 RNA suppression <200 copies/mL was maintained in all patients.

The high virologic response rate and low rate of treatment discontinuation observed with 3D+RBV in treatment-naïve and treatment-experienced GT1 HCV/HIV-1 co-infected patients with or without cirrhosis is consistent with those in HCV GT1-monoinfected populations receiving this regimen.

TABLE 6
Patients Baseline Profiles

| Baseline Demographics and | 12-Week 3D + RBV | |
|---|---|---|
| Characteristics, n (%) | N = 31 | |
| Age (yrs), mean (range) | 50.9 | (38-66) |
| Sex, Male | 29 | (93.5) |
| Race, Black | 7 | (22.6) |
| HCV GT1a | 27 | (87.1) |
| IL28B Non-CC | 26 | (83.9) |
| Prior Treatment Experience | | |
| Naïve | 20 | (64.5) |
| Relapser | 1 | (3.2) |
| Partial Responder | 5 | (16.1) |
| Null Responder | 5 | (16.1) |
| Cirrhosis | 6 | (19.4) |
| HIV-1 ART Regimen | | |
| Atazanavir | 16 | (51.6) |
| Raltegravir | 15 | (48.4) |

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method of treatment for a patient infected with HCV genotype 1b, comprising administering Compound 1 or a pharmaceutically acceptable salt thereof, and Compound 2 or a pharmaceutically acceptable salt thereof, to said patient, wherein said treatment does not include administration of either interferon or ribavirin to said patient, and said treatment lasts from 8 to 12 weeks, and wherein Compound 1 or the salt thereof is administered with ritonavir.

2. The method of claim 1, wherein said treatment lasts 8 weeks.

3. The method of claim 1, wherein said treatment lasts 12 weeks.

4. The method of claim 1, comprising administered 150 mg Compound 1, 100 mg ritonavir, and 25 mg Compound 2 to said patient once daily.

5. The method of claim 4, wherein Compound 1, ritonavir and Compound 2 are co-formulated in a solid dosage form.

6. The method of claim 5, wherein said patient is a treatment-naïve patient.

7. The method of claim 5, wherein said patient is an interferon null responder.

8. A method of treatment for a patient infected with HCV genotype 4, comprising administering Compound 1 or a pharmaceutically acceptable salt thereof, and Compound 2 or a pharmaceutically acceptable salt thereof, to said patient, wherein said treatment does not include administration of interferon to said patient, and said treatment lasts from 8 to 12 weeks, and wherein Compound 1 or the salt thereof is administered with ritonavir.

9. The method of claim 8, wherein said treatment lasts 8 weeks.

10. The method of claim 8, wherein said treatment lasts 12 weeks.

11. The method of claim 8, further comprising administered ribavirin to said patient.

12. The method of claim 8, wherein said treatment does not include administration of ribavirin to said patient.

13. The method of claim 8, comprising administered 150 mg Compound 1, 100 mg ritonavir, and 25 mg Compound 2 to said patient once daily.

14. The method of claim 13, wherein Compound 1, ritonavir and Compound 2 are co-formulated in a solid dosage form.

15. The method of claim 14, wherein said patient is a treatment-naïve patient.

16. The method of claim 14, wherein said patient is an interferon null responder.
